(19) United States
(12) Patent Application Publication
(10) Pub. No.: US 2010/0285115 A1
(43) Pub. Date: Nov. 11, 2010

(54) Novel Antiretroviral Combination

(75) Inventors: Amar Lulla, Mumbai (IN); Geena Malhotra, Mumbai (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 12/740,255

(22) PCT Filed: Oct. 28, 2008
(86) PCT No.: PCT/GB2008/003651

§ 371 Date: Jul. 15, 2010

(30) Foreign Application Priority Data
Oct. 29, 2007 (IN) 2141/MUM/2007

(51) Int. Cl. A61K 31/4418; A61K 9/20; A61K 9/48; A61P 31/18

(52) U.S. Cl. 424/452; 514/357; 424/464; 424/465

(57) ABSTRACT

The invention relates to pharmaceutical compositions containing a combination of atazanavir and ritonavir, to methods of making them, and their use in medicine.

### CROSS-REFERENCE TO RELATED APPLICATIONS

This application is a filing under 35 U.S.C. 371 of International Application No. PCT/GB2008/003651 filed Oct. 28, 2008, entitled “Novel Antiretroviral Combination,” claiming priority of Indian Patent Application No. 2141/MUM/2007 filed Oct. 29, 2007, which applications are incorporated by reference herein in their entirety.

### FIELD OF INVENTION

The present invention relates to a novel oral pharmaceutical antiretroviral combination in particular, to a novel oral pharmaceutical antiretroviral composition and a process for manufacturing the same.

### BACKGROUND OF INVENTION

The human immunodeficiency virus (HIV) is a pathogenic retrovirus and the causative agent of Acquired Immune Deficiency Syndrome (AIDS) and related disorders (Barre-Sinossi, F. et al., 1983, Science 220:868-870; Gallo, R. et al., 1984, Science 224:500-503). There are at least two distinct types of HIV: HIV-1 (Barre-Sinossi, F. et al., 1983, Science 220:868-870; Gallo, R. et al., 1984, Science 224:500-503) and HIV-2 (Clavel, F. et al., 1986, Science 223:343-346; Guyader, M. et al., 1987, Nature 326:662-669). Further, a large amount of genetic heterogeneity exists within populations of each of these types. Infection of human CD-4+T-lymphocytes with an HIV virus leads to depletion of the cell type and eventually to opportunistic infections, neurological dysfunctions, neoplastic growth, and untimely death.

HIV is a member of the lentivirus family of retroviruses (Teich, N. et al., 1984; RNA Tumor Viruses, Weiss, R. et al., eds., CSH-press, pp. 949-956). Retroviruses are small enveloped viruses that contain a diploid, single-stranded RNA genome, and replicate via a DNA intermediate produced by a virally-encoded reverse transcriptase, an RNA-dependent DNA polymerase (Varmus, H., 1988, Science 240:1427-1439). Other retroviruses include, for example, oncogenic viruses such as human T-cell leukemia viruses (HTLV-1, -II, -III), and feline leukemia virus. The HIV viral particle consists of a viral core, made up of proteins designated p24 and p18. The viral core contains the viral RNA genome and those enzymes required for replicative events. Myristylated gag protein forms an outer viral shell around the viral core, which is, in turn, surrounded by a lipid membrane envelope derived from the infected cell membrane.

The HIV envelope surface glycoproteins are synthesized as a single 160 kD precursor protein which is cleaved by a cellular protease during viral budding into two glycoproteins, gp41 and gp120. gp41 is a transmembrane protein and gp120 is an extracellular protein which remains noncovalently associated with gp41, possibly in a trimeric or multimeric form (Hammerwskjold, M. and Rekosh, D., 1989, Biochem. Biophys. Acta 989:269-280).

Attention is also being given to the development of vaccines for the treatment of HIV infection. The HIV-1 envelope proteins (gp160, gp120, gp41) have been shown to be the major antigens for anti-HIV antibodies present in AIDS patients (Barin et al., 1985, Science 228:1094-1096). Thus far, these proteins seem to be the most promising candidates to act as antigens for anti-HIV development. To this end, several groups have begun to use various portions of gp160, gp120, and/or gp41 as immunogenic targets for the host immune systems. See, for example, Ivanoff, L. et al., U.S. Pat. No. 5,141,867; Saith, G. et al., WO 92/22, 654; Schafferman, A., WO 91/09,872; Formoso, C. et al., WO 90/07,119. Clinical results concerning these candidate vaccines, however, still remain far in the future.

U.S. Pat. No. 5,541,206 (Kempf, Dale J., et al.) discloses the synthesis and use of ritonavir to inhibit HIV infection.

U.S. Pat. No. 5,674,882 (Kempf, Dale J., et al.) discloses the use of ritonavir in combination with one or more HIV protease inhibitors to inhibit an HIV infection.

U.S. Pat. No. 6,037,157 (Norbeck, Daniel W., et al.) and WO 97/01349 (Norbeck, Daniel W., et al.) discloses the use of ritonavir to enhance the pharmacokinetics of compounds metabolized by cytochrome P450 monooxygenase.

U.S. Pat. No. 5,484,801 (A1-Razzak, Laman A., et al.) discloses a liquid dosage form of ritonavir for oral administration.

WO95/07696 (A1-Razzak, Laman A., et al.) discloses an encapsulated solid or semi-solid dosage form for ritonavir.

US 20070208009 (Hoetelmans Richard Marinus W., et al.) discloses a combination comprising tenofovir, ritonavir and darunavir for treatment or prevention of HIV infections.

U.S. Pat. No. 4,950,652 (Carter, William A., et al.) discloses combinations of double stranded RNA's with antiviral agents such as interferon, AZT, and phosphonoformate to treat viral infections.

WO2005007070 (Pacheco, Ogari, et al.) discloses compositions comprising a solution of one or two HIV protease inhibitors in a combination of pharmaceutical acceptable organic solvents, a surfactant, and a bioavailability enhancer.

U.S. Pat. No. 5,077,280 (Sommadossi, Jean-Pierre, et al.) discloses a combination therapy combining a pyrimidine nucleoside analog and a uridine phosphorylase inhibitor for the treatment of HIV.

U.S. Pat. No. 6,506,555 (Andre, Patrice, et al.) discloses a medicament having a compound selected from ritonavir, saquinavir or the salts in association with a pharmaceutically acceptable vehicle which modulates proteasome.

The disclosures of all these patents and patent applications are herein incorporated by reference.

None of the current AIDS treatments have proven to be totally effective in treating and/or reversing the disease till date. In addition, many of the compounds currently used to treat AIDS cause adverse side effects including low platelet count, renal toxicity, and bone marrow cytopenia.

Some drugs and, in particular, some HIV protease inhibitors are metabolized by cytochrome P450 monooxygenase, leading to unfavorable pharmacokinetics and hence require more frequent and higher doses, although administration of such drugs with an agent that inhibits metabolism by cytochrome P450 monooxygenase will improve the pharmacokinetics (i.e., increase half-life, increase the time to peak plasma concentration, increase blood levels) of the drug.

Moreover, combination therapy is potentially problematic given the high toxicity of most anti-HIV therapeutics and their low level of effectiveness.

Thus, there is a need for a combination therapy which is effective yet non-toxic both for treatment-naïve and treatment-experienced patients.

Surprisingly, the present inventors have found that a selective combination of atazanavir and ritonavir with pharmaceutically acceptable excipients and using simpler manufacturing processes achieves the desired formulation.

Further, the selective combination of atazanavir and ritonavir with pharmaceutically acceptable excipients potentially increases the treatment potency particularly against drug-resistant HIV-1 strains, without significantly raising the risk for toxicity in treatment-naïve and treatment-experienced patients.

### OBJECT OF THE INVENTION

An object of the present invention is to provide an oral antiretroviral composition comprising a novel antiretroviral combination which may be administered simultaneously, separately or sequentially.

Another object of the present invention is to provide an oral antiretroviral composition comprising a novel antiretroviral combination with increased drug exposure and high treatment potency.

Another object of the present invention is to provide an oral antiretroviral composition comprising a novel antiretroviral combination which is highly potent against wild-type and multidrug-resistant HIV strains.

Still another object of the present invention is to provide an oral antiretroviral composition with ease of manufacture.

### SUMMARY OF THE INVENTION

Broadly, the invention relates to a composition comprising:
(i) a first protease inhibitor, or a pharmaceutically acceptable salt thereof;
(ii) a second protease inhibitor or a pharmaceutically acceptable salt thereof, and/or a cytochrome P450 inhibitor, or its pharmaceutically acceptable salt.
(iii) Optionally, one or more pharmaceutically acceptable excipients.

The first protease inhibitor is preferably atazanavir or a pharmaceutically acceptable salt thereof.

The second protease inhibitor is preferably ritonavir or a pharmaceutically acceptable salt thereof. The cytochrome P450 inhibitor is preferably ritonavir or a pharmaceutically acceptable salt thereof. It will therefore be appreciated that the second protease inhibitor may be the same as the cytochrome P450 inhibitor.

The composition may include one or more additional protease inhibitors or one or more additional cytochrome P450 inhibitors.

It will be appreciated that the first protease inhibitor, in particular atazanavir, may be provided as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, or a pharmaceutically acceptable prodrug thereof.

It will further be appreciated that the second protease inhibitor, in particular ritonavir, may be provided as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph thereof, or a pharmaceutically acceptable prodrug thereof.

It will be further appreciated that the cytochrome P450 inhibitor, in particular ritonavir, may be provided as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, or a pharmaceutically acceptable prodrug thereof.

According to one aspect of the present invention there is provided a pharmaceutical combination of one or more protease inhibitors, and optionally, one or more pharmaceutically acceptable excipients. The combination may include one or more cytochrome P450 inhibitors.

According to another aspect of the present invention there is provided an oral antiretroviral composition comprising one or more protease inhibitors, and optionally, one or more pharmaceutically acceptable excipients, in a single dose regimen. The composition may include one or more cytochrome P450 inhibitors.

According to another aspect of the present invention there is provided a process of manufacturing the oral antiretroviral composition comprising one or more protease inhibitors, and optionally one or more pharmaceutically acceptable excipients. The composition may include one or more cytochrome P450 inhibitors.

According to yet another aspect of the present invention, there is provided an oral antiretroviral combination of one or more protease inhibitors, and optionally, one or more pharmaceutically acceptable excipients, for use in the treatment against HIV strains. The combination may include one or more cytochrome P450 inhibitors.

According to a further aspect of the present invention, there is provided an oral antiretroviral combination of one or more protease inhibitors, and optionally, one or more pharmaceutically acceptable excipients, for use in the manufacture of a medicament used in the treatment against HIV strains. The combination may include one or more cytochrome P450 inhibitor(s) or pharmaceutically acceptable salts, pharmaceutically acceptable solvates, pharmaceutically acceptable enantiomers, pharmaceutically acceptable derivatives, pharmaceutically acceptable polymorphs, or pharmaceutically acceptable prodrugs.

In one preferred embodiment, the invention utilises at least two protease inhibitors. Preferably, one protease inhibitor is ritonavir or a pharmaceutically acceptable salt thereof, and the other is atazanavir or a pharmaceutically acceptable salt thereof.

As mentioned above, it will be appreciated that each of the ritonavir and atazanavir may be provided as a pharmaceutically acceptable solvate, a pharmaceutically acceptable enantiomer, a pharmaceutically acceptable derivative, a pharmaceutically acceptable polymorph, or a pharmaceutically acceptable prodrug thereof.

In another preferred embodiment, the invention utilises at least one protease inhibitor and at least one cytochrome P450 inhibitor. Preferably, the invention utilizes ritonavir as the P450 inhibitor. Ritonavir can act as both a protease inhibitor and a cytochrome P450 inhibitor.

### DETAILED DESCRIPTION OF THE INVENTION

As discussed above and hereinafter, the present invention in one embodiment relates to a novel anti retroviral combination of one or more protease inhibitors preferably atazanavir, or a pharmaceutically acceptable salt thereof in a single dose regimen. When the two protease inhibitors, i.e., atazanavir and ritonavir are given in combination, the drug exposure to atazanavir is increased leading to maximum concentration whereby the pharmacokinetic principles, i.e., Cₘᵢₙ and AUC for the combination on this regimen are 5 times and 3 times higher, respectively, without substantially increasing the maximum plasma concentration (WO 97/01349 by Norbeck, Daniel W., et al.). When the combination is administered, there is reduction in total fasting triglyceride and cholesterol levels and hence doesn't require lipid lowering therapy unlike the case when atazanavir is administered alone.

Ritonavir has poor oral bioavailability when manufactured in solid dosage form such as tablet and is available in a soft gelatin capsule (U.S. Pat. No. 6,458,818 to Lipari. John, et at). Hence making ritonavir and its combinations with other actives solid dosage form such as tablet or hard gelatin capsule is challenging. We have found a way to solve this problem by providing the drugs in a multilayer dosage form, in which the ritonavir layer has been produced by a hot melt extrusion process, which can convert ritonavir into amorphous form while the atazanavir layer may be prepared by a conventional process, such as wet granulation or direct compression. We have found that a multilayer tablet produced in this way, solves the formulation problem as discussed above.

It will be well appreciated that the combination of atazanavir and ritonavir or their pharmaceutically acceptable salts, pharmaceutically acceptable solvates, pharmaceutically acceptable enantiomers, pharmaceutically acceptable derivatives, pharmaceutically acceptable polymorphs, or pharmaceutically acceptable prodrugs may further comprise one or more pharmaceutically acceptable excipients yielding the desired oral antiretroviral composition.

The term “atazanavir” and “ritonavir” is mentioned in the description as well as the claims in a broad sense to include not only atazanavir and ritonavir, per se, but also its pharmaceutically acceptable salts, pharmaceutically acceptable solvates, pharmaceutically acceptable enantiomers, pharmaceutically acceptable derivatives, pharmaceutically acceptable polymorphs, or pharmaceutically acceptable prodrugs thereof.

In each embodiment, suitably, the formulations according to the invention are presented in solid dosage form, conveniently in unit dosage form, and include dosage form suitable for oral and buccal administration.

The preferred dosage form for the composition according to the invention is a solid unit dosage form such as tablet or capsule.

A preferred formulation according to the invention is in tablet dosage form wherein the drug combination viz. atazanavir or its pharmaceutically acceptable salts, and ritonavir or its pharmaceutically acceptable salts, and optionally comprises one or more pharmaceutically acceptable excipients.

It will be understood, however, that specific dose level and frequency of dosage of the combination according to the invention for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

According to the preferred embodiment, the combination, according to the invention, will be administered in the following daily dosages; atazanavir about 70 mg to 400 mg, preferably 300 mg, and ritonavir about 20 mg to 200 mg, preferably 100 mg. These dosage ranges are suitable for adults.

It is well known in the art that a tablet formulation is the preferred solid dosage form due to its greater stability, less risk of chemical interaction between different medicaments, smaller bulk, accurate dosage, and ease of production.

According to the preferred embodiment, the formulation is administered in a single unit dosage form wherein each active ingredient is provided in a separate layer of a multilayer tablet.

According to a particularly advantageous embodiment, the formulation may be administered as a bilayer tablet wherein each layer separately contains a respective one of the active ingredients and optional pharmaceutically acceptable excipients.

According to yet another embodiment, the formulation may be seal coated. According to yet another embodiment, the formulation may be seal coated and further film coated.

The present invention may be manufactured through various techniques or processes known in the art which includes, but are not limited to, direct compression, melt granulation, melt extrusion, spray drying and solution evaporation.

According to an especially preferred embodiment, the invention may be processed through hot melt extrusion technique which involves hot melt extrusion of one or more drug(s) with one or more polymer(s), wherein the polymer comprises of one or more water insoluble polymer(s) and/or a combination of one or more water soluble polymer(s) and one or more water insoluble polymer(s) wherein the drug:polymer ratio ranges from 1:1 to 1:6.

In general terms, the process of hot melt extrusion is carried out in the conventional extruders as known to a person skilled in the art.

The melt-extrusion process comprises the steps of preparing a homogeneous melt of one or more drugs, the polymer and, optionally, one or more excipients, and cooling the melt until it solidifies. “Melting” means a transition into a liquid or rubbery state in which it is possible for one component to get embedded homogeneously in the other.

Typically, one component will melt and the other components will dissolve in the melt thus forming a solution. Melting usually involves heating above the softening point of the polymer. The preparation of the melt can take place in a variety of ways. The mixing of the components may take place before, during, or after the formation of the melt. For example, the components can be mixed first and then melt extruded or be simultaneously mixed and melt extruded. Usually, the melt is homogenized in order to disperse the active ingredients efficiently. Also, it may be convenient first to melt the polymer and then to mix in and homogenize the active ingredients.

Usually, the melt temperature is in the range of about 50° C. to about 200° C., preferably from about 70° C. to about 180° C., more preferably from about 80° C. to about 150° C.

Suitable extruders include single screw extruders, intermeshing screw extruders, or else multiscrew extruders, preferably twin screw extruders, which can be co-rotating or counter-rotating and, optionally, be equipped with kneading disks. It will be appreciated that the working temperatures will also be determined by the kind of extruder or the kind of configuration within the extruder that is used.

The extrudates can be in the form of beads, granulates, tube, strand or cylinder and this can be further processed into any desired shape.

The term ‘extrudates’ as used herein refers to solid product solutions, solid dispersions and glass solutions of one or more drugs with one or more polymers and optionally pharmaceutically acceptable excipients.

According to a preferred embodiment, a powder blend of the one or more active drug(s) and polymers and optionally pharmaceutical excipients are transferred by a rotating screw of a single screw extruder through the heated barrel of an extruder whereby the powder blend melts and molten solution product is collected on a conveyor where it is allowed to cool to form an extrudate. Shaping of the extrudate can be conveniently be carried out by a calender with two counter-rotating rollers with mutually matching depressions on their surface.

A broad range of tablet forms can be attained by using rollers with different forms of depressions. Alternatively, the extrudate is cut into pieces after solidification and can be further processed into suitable dosage forms. More preferably the extrudates thus finally obtained from the above process are then milled and ground to granules by the means known to a person skilled in the art.

Further, hot melt extrusion is a fast, continuous, single pot manufacturing process, which does not require further drying or discontinuous process steps; provides short thermal exposure of the active material, and therefore allows processing of heat sensitive actives; allows reduction of the process temperatures by addition of plasticizers; and has comparatively lower investment for equipment as against other processes. The entire process is anhydrous and the intense mixing and agitation of the powder blend that occur during processing contribute to a very homogenous extrudate(s).

In one aspect, the preferred embodiment in accordance with the present invention may comprise one or more protease inhibitors and one or more water insoluble polymers which are melt extruded by the process as described herein, where a powder blend of one or more protease inhibitors most preferably atazanavir or a pharmaceutically acceptable salt thereof, and/or ritonavir or a pharmaceutically acceptable salt thereof, and polymer or a combination thereof and other excipients which may comprise suitable bulking agents and flavourants. These are so processed to form a powder blend which is transferred through the heated barrel of the extruder, whereby the powder blend melts and molten solution product is collected on a conveyor whereby it is allowed to cool and form an extrudate.

Alternatively, the extrudate is cut into pieces after solidification and can be further processed into suitable dosage forms. More preferably the extrudates thus finally obtained from the above process are then milled and ground to granules by the means known to a person skilled in the art.

In another aspect, the preferred embodiment in accordance with the present invention may comprise one or more protease inhibitors and a combination of one or more water insoluble polymer and one or more water soluble polymer which are melt extruded by the process as described herein, where a powder blend of one or more protease inhibitors, i.e., atazanavir or its pharmaceutically acceptable salts, and/or ritonavir or its pharmaceutically acceptable salts, and a combination of water soluble polymer(s) and water insoluble polymer(s) and other excipients which may comprise suitable bulking agents, plasticizer and flavourants.

In another aspect, the preferred embodiment in accordance with the present invention may comprise one or more protease inhibiting drug/s, one or more cytochrome P450 inhibitors and a combination of one or more water insoluble polymer and one or more water soluble polymer which are melt extruded by the process as described herein, where a powder blend of protease inhibitor most preferably atazanavir or its pharmaceutically acceptable salts, and/or cytochrome P450 inhibitor, most preferably ritonavir or its pharmaceutically acceptable salts, and a combination of water soluble polymer(s) and water insoluble polymer(s) and other excipients which may comprise suitable bulking agents, plasticizer and flavourants.

These are so processed to form a powder blend which is transferred through the heated barrel of the extruder, whereby the powder blend melts and molten solution product is collected on a conveyor whereby it is allowed to cool and form an extrudate.

Alternatively, the extrudate is cut into pieces after solidification and can be further processed into suitable dosage forms. More preferably the extrudates thus finally obtained from the above process are then milled and ground to granules by the means known to a person skilled in the art.

The water soluble polymers that can be used, according to the present invention, comprise homopolymers and co-polymers of N-vinyl lactams, especially homopolymers and co-polymers of N-vinyl pyrrolidone, e.g., polyvinylpyrrolidone (PVP), co-polymers of PVP and vinyl acetate such as Copovidone (e.g. Kollidon VA 64), co-polymers of N-vinyl pyrrolidone and vinyl acetate or vinyl propionate, cellulose esters and cellulose ethers, high molecular polyalkylene oxides such as polyethylene oxide and polypropylene oxide and co-polymers of ethylene oxide and propylene oxide. The water soluble polymer component is preferably present in the range wherein the ratio of drug to polymer is from 1:1 to 1:6.

The water insoluble polymer that can be used, according to the present invention, comprises of acrylic copolymers, e.g., Eudragit E100 or Eudragit EPO; Eudragit L30D-55, Eudragit FS30D, Eudragit RL30D, Eudragit RS30D, Eudragit NE30D, Acryl-Eze (Colorcon Co.); polyvinylacetate, for example, Kollicoat SR 30D (BASF Co.); cellulose derivatives such as ethylcellulose, cellulose acetate, e.g., Surelease (Colorcon Co.), Aquacoat ECD and Aquacoat CPD (FMC Co.). Most preferably, the water insoluble polymer is being Eudragit E100 and the water insoluble polymer component is preferably present in the range wherein the ratio of drug to polymer is from 1:1 to 1:6.

If both a water insoluble polymer and a water soluble polymer are used, then it is preferred that the ratio of drug to total amount of polymer is from 1:1 to 1:6.

Plasticizers can be incorporated depending on the polymer and the process requirement. These, advantageously, when used in the hot melt extrusion process decrease the glass transition temperature of the polymer. Plasticizers also help in reducing the viscosity of the polymer melt and thereby allow for lower processing temperature and extruder torque during hot melt extrusion. Examples of plasticizers which can be used in the present invention, include, but are not limited to, polysorbates such as sorbitan monolaurate (Span 20), sorbitan monopalmitate, sorbitan monostearate, sorbitan monoisostearate; citrate ester type plasticizers like triethyl citrate, citrate phthalate; propylene glycol; glycerin; low molecular weight polyethylene glycol (PEG) with molecular weights ranging from 400 to 8000, such as PEG 600, PEG 1000, PEG 1500, PEG 3000, PEG 4000, PEG 6000 or PEG 7000; polyoxyethylene castor oil derivatives such as polyoxyl castor oil, polyoxyl 35 castor oil (Cremophor EL and Cremophor ELP), polyoxyl 40 hydrogenated castor oil (Cremophor RH 40), polyoxyl 40 hydrogenated castor oil (Cremophor RH 60), triacetin; dibutyl sebacate, tributyl sebacate; dibutyltartrate, dibutyl phthalate. It is preferably present in an amount ranging from 0% to 10% to the weight of polymer.

According to a preferred embodiment, the present invention may comprise one or more disintegrating agents which may include, but are not limited to, croscarmellose sodium, crospovidone, sodium starch glycolate, corn starch, potato starch, maize starch and modified starches, calcium silicates, low substituted hydroxy-propylcellulose. The amount of disintegrant is preferably in the range of 5% to 35% by weight of the composition.

According to a preferred embodiment, the present invention may further comprise one or more bulking agents which may include, but are not limited to, a saccharide, including a monosaccharide, a disaccharide, a polysaccharide or a sugar alcohol such as arabinose, lactose, dextrose, sucrose, fructose, maltose, mannitol, erythritol, sorbitol, xylitol lactitol, and other bulking agents such as powdered cellulose, microcrystalline cellulose, purified sugar and derivatives thereof. The formulation may incorporate one or more of the above bulking agents. Preferably, lactose and microcrystalline cellulose forms the bulking agent. The amount of the bulking agent is preferably in the range of 15% to 70% by weight of the composition.

Accordingly, the present invention may further incorporate one or more lubricants and glidants which may include, but are not limited to, stearic acid and its derivatives or esters like sodium stearate, magnesium stearate and calcium stearate and the corresponding esters such as sodium stearate fumarate; talc and silicon dioxide, respectively. The amount of lubricant and/or glidant is preferably in the range of 0.25% to 5% by weight of the composition.

According to another embodiment, the present invention may further involves a manufacturing process to obtain a single unitary dosage form, i.e., wherein the or each drug is processed by the techniques as discussed above and finally compacted to yield a single dosage form. Preferably, atazanavir or a pharmaceutically acceptable salt thereof, in combination with one or more optional excipients and ritonavir or a pharmaceutically acceptable salt thereof, in combination with one or more optional excipients may be processed with the techniques as discussed above separately and may be combined to form single unitary dosage form. Preferably, the atazanavir, optional excipients, is compacted and compressed into a tablet and the ritonavir, with optional excipients is compacted and compressed into tablet and finally each individual layer is compressed into a bilayer tablet. More preferably, the tablet is seal coated. Most preferably, the tablet is seal coated and finally film coated. The formulation may be coated with Ready colour mix systems (such as Opadry colour mix systems).

According to yet another embodiment, the present invention may be formulated wherein the or each drug, preferably, atazanavir or a pharmaceutically acceptable salt thereof, and one or more optional excipients, is processed through wet granulation, direct compression and the like as mentioned above and ritonavir or a pharmaceutically acceptable salt thereof is processed through melt granulation, melt extrusion, and the like as mentioned above.

Preferably, atazanavir or a pharmaceutically acceptable salt thereof, is mixed with intragranular excipients which include, but are not limited to, diluents, disintegrants and granulated with water, sieved, sifted and lubricated, and dried. Alternatively, the dried granules may be compressed into tablets.

Preferably, ritonavir or a pharmaceutically acceptable salt thereof, and one or more excipients which include, but are not limited to, polymers (i.e., either water soluble or water insoluble or mixture thereof), one or more plasticizer, one or more disintegrants, one or more lubricants and glidants are extruded through hot melt extrusion technique wherein extrudates are obtained which can be molded into desired shapes that can be filled in sachets or can be granulated. Alternatively, the granules may be compressed into tablets.

According to a preferred embodiment, the granules (comprising the individual actives) as obtained above may be further mixed, sieved, sifted and compressed into a single tablet or may be filled into capsules or sachets, or the granules may be administered directly. Alternatively, the tablet may be seal coated and finally film coated.

According to a preferred embodiment, the or each granules (comprising the individual actives) as obtained above may be individually compressed into two tablets and finally compacted and compressed into a bilayer tablet. Alternatively, the tablet may be seal coated and finally film coated.

The formulation can be coated with Ready colour mix systems (such as Opadry colour mix systems).

According to another aspect of the invention, there is provided a pharmaceutical composition comprising a solid unit multilayer dosage form comprising: (i) a first layer containing ritonavir, which has been made by a hot melt extrusion process; and (ii) a second layer containing atazanavir or a pharmaceutically acceptable salt thereof, which is made by suitable techniques known in the art.

The present invention further features methods of treating HIV infection. These methods comprise administering to a human in need of such treatment a dosage form of the present invention.

### EXAMPLES

The following examples are for the purpose of illustration of the invention only and is not intended in any way to limit the scope of the present invention.

### Example 1

Formula:

| | QUANTITY |
|---|---|
| INGREDIENTS | (mg/tab) |
| ATAZANAVIR LAYER | |
| Atazanavir sulphate equivalent | 341.70 |
| to 300 mg Atazanavir | |
| Lactose monohydrate | 82.00 |
| Crospovidone | 14.00 |
| Yellow Iron Oxide | 0.30 |
| Magnesium stearate | 2.00 |
| Purified water | q.s. |
| RITONAVIR LAYER | |
| Drug Premix | |
| Ritonavir | 100.00 |
| Colloidal silicon dioxide | 5.00 |
| Polymer Premix | |
| Kollidon VA 64 | 400.00 |
| Span 20 | 40.00 |
| Blending | |
| Crospovidone | 50.00 |
| Colloidal silicon dioxide | 5.00 |
| Microcrystalline cellulose | 40.00 |
| Lubrication | |
| Sodium stearyl fumarate | 10.00 |
| Seal Coating | |
| Opadry AMB OY- B -29000 translucent | 5.00 |
| Purified water | q.s. |
| Film Coating | |
| Opadry 04F52201 yellow | 15.00 |
| Purified water | q.s. |
| TOTAL | 1110.00 |

Process:

(1) Atazanavir sulphate was mixed with pre-sieved and pre-sifted amounts of lactose monohydrate, crospovidone, yellow iron oxide, magnesium stearate and granulated with purified water.
(2) Ritonavir with small amount of colloidal silicon dioxide was sifted and mixed together with Kollidon VA 64 and Span 20 in a mixer.
(3) The contents obtained in (2) were mixed and finally subjected to hot melt extrusion (HME) wherein the melting temperature for the extrusion process ranges from 50 to 200° C., with the molten mass thus obtained being collected on a conveyor where it was cooled to form extrudates and these extrudates on further milling were converted into granules which was followed by addition of crospovidone, colloidal silicon dioxide and microcrystalline cellulose and further lubricated with sodium stearyl fumarate.
(4) The granules obtained in (1) and (3) were compressed together to form a bilayer tablet which was then seal coated and finally film coated.

### Example 2

Formula:

| | QUANTITY |
|---|---|
| INGREDIENTS | (mg/tab) |
| ATAZANAVIR LAYER | |
| Dry Mix | |
| Atazanavir | 75.00 |
| Lactose monohydrate | 51.40 |
| Crospovidone | 4.00 |
| Yellow Iron Oxide | 0.05 |
| Binder | |
| Purified water | q.s. |
| Lubrication | |
| Crospovidone | 4.00 |
| Magnesium stearate | 0.55 |
| RITONAVIR LAYER | |
| Drug Premix | |
| Ritonavir | 25.00 |
| Colloidal silicon dioxide | 1.25 |
| Crospovidone | 25.00 |
| Polymer Premix | |
| Copovidone (Kollidon VA 64) | 100.00 |
| Tween 20 | 10.00 |
| Blending | |
| Crospovidone | 25.00 |
| Colloidal silicon dioxide | 2.50 |
| Microcrystalline cellulose | 108.75 |
| Lubrication | |
| Sodium stearyl fumarate | 2.50 |
| Total | 300.00 |

Process:

(1) Atazanavir sulphate was mixed with pre-sieved and pre-sifted amounts of lactose monohydrate, crospovidone, yellow iron oxide, granulated with purified water and lubricated with crospovidone and magnesium stearate.
(2) Ritonavir with small amount of colloidal silicon dioxide was sifted and mixed together with Copovidone (Kollidon VA 64) in a mixer.
(3) Mixture of Copovidone and Tween 20 were blended with crospovidone, colloidal silicon dioxide and microcrystalline cellulose to form polymer premix.
(4) The contents obtained in (2) and (3) were mixed and finally subjected to hot melt extrusion (HME) wherein the melting temperature for the extrusion process ranges from 50 to 200° C., with the molten mass thus obtained being collected on a conveyor where it was cooled to form extrudates and these extrudates on further milling were converted into granules which was followed by addition of crospovidone, colloidal silicon dioxide, and microcrystalline cellulose and further lubricated with sodium stearyl fumarate.
(5) The granules obtained in (1) and (4) were compressed together to form a bilayer tablet which was then seal coated and finally film coated.

### Example 3

Formula:

| | QUANTITY |
|---|---|
| INGREDIENTS | (mg/capsule) |
| ATAZANAVIR | |
| Atazanavir sulphate equivalent | 341.70 |
| to 300 mg Atazanavir | |
| Lactose monohydrate | 82.00 |
| Crospovidone | 14.00 |
| Yellow Iron Oxide | 0.30 |
| Magnesium stearate | 2.00 |
| Purified water | q.s. |
| RITONAVIR | |
| Drug Premix | |
| Ritonavir | 100.00 |
| Colloidal silicon dioxide | 6.90 |
| Polymer Premix | |
| Copovidone (Kollidon VA 64) | 493.10 |
| Polyoxyl 40 hydrogenated castor oil | 100.00 |
| Blending & Lubrication | |
| Colloidal silicon dioxide | 13.90 |
| Dibasic calcium phosphate (Anhydrous) | 136.10 |
| Total | 1290.00 |

Process:

(1) Atazanavir sulphate was mixed with pre-sieved and pre-sifted amounts of lactose monohydrate, crospovidone, yellow iron oxide, magnesium stearate and granulated with purified water.
(2) Ritonavir with small amount of colloidal silicon dioxide was sifted and mixed together with Kollidon VA 64 and polyoxyl 40 hydrogenated castor oil in a mixer.
(3) The contents obtained in (2) were mixed and finally subjected to hot melt extrusion (HME) wherein the melting temperature for the extrusion process ranges from 50 to 200° C., with the molten mass thus obtained being collected on a conveyor where it was cooled to form extrudates and these extrudates on further milling were converted into granules which was followed by addition of dibasic calcium phosphate (anhydrous) and further lubricated with colloidal silicon dioxide.
(4) The granules obtained in (1) and (3) were filled into capsules and optionally with other pharmaceutically acceptable excipients.

### Example 4

Stability study data of Atazanavir and Ritonavir tablet:

| | Condition Parameters | | | | | |
|---|---|---|---|---|---|---|
| | | | 25° C./60% RH | | 40° C./75% RH | |
| | Initial | | 2 M | | 2 M | |
| Assay (%) (Limit 90-110% | Atazanavir | Ritonavir | Atazanavir | Ritonavir | Atazanavir | Ritonavir |
| of Labelled amount) | 100.3 | 99.5 | 99.0 | 99.3 | 98 | 96 |

Impurities
Ritonavir Layer

| | | | |
|---|---|---|---|
| Total (Limit- | 0.98 | 0.90 | 1.14 |
| NMT 2.50%) | | | |

Atazanavir Layer

| | | | |
|---|---|---|---|
| Total- (Limit- | 0.45 | 0.41 | 0.47 |
| NMT 1.0%) | | | |

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a polymer” includes a single polymer as well as two or more different polymers; reference to a “plasticizer” refers to a single plasticizer or to combinations of two or more plasticizer, and the like.

1. A pharmaceutical composition comprising a solid unit dosage form comprising:
(i) ritonavir or a pharmaceutically acceptable salt thereof; and
(ii) atazanavir or a pharmaceutically acceptable salt thereof.

2. The pharmaceutical composition according to claim 1, which is a tablet formulation comprising said ritonavir in a first layer of the formulation and said atazanavir in a second layer of the formulation.

3. The pharmaceutical composition according to claim 1, further comprising a water insoluble polymer and/or a water soluble polymer.

4. The pharmaceutical composition according to claim 3, wherein the ratio of the weight of the ritonavir or atazanavir to the weight of the polymer is from 1:1 to 1:6.

5. The pharmaceutical composition according to claim 3, wherein the polymer is present in at least the layer containing the ritonavir.

6. The pharmaceutical composition according to claim 1, further comprising at least one pharmaceutically acceptable excipient.

7. The pharmaceutical composition according to claim 6, wherein the excipient includes a plasticizer.

8. The pharmaceutical composition according to claim 1, wherein the atazanavir is present in an amount from 70 to 400 mg.

9. The pharmaceutical composition according to claim 1, wherein the ritonavir is present in an amount from 20 to 200 mg.

10. The pharmaceutical composition according to claim 1 for use in treating HIV or AIDS.

11. The pharmaceutical composition according to claim 2, wherein the layer containing the ritonavir is obtainable by hot melt extruding said ritonavir with a polymer.

12. A method of treating HIV or AIDS comprising administering a therapeutically effective amount of a pharmaceutical composition as defined in claim 1.

13. A method of making a pharmaceutical composition as defined in claim 2, when dependent on claim 2, comprising hot melt extruding the ritonavir to form an extrudate, then formulating the extrudate into said first layer; formulating said atazanavir—into said second tablet layer; and combining said first and second layer to provide a single unitary multiple layer tablet formulation.

14. The method according to claim 13, wherein the ritonavir is mixed with a water soluble polymer and/or a water insoluble polymer prior the hot melt extrusion step.

15. The method according to claim 13, wherein the atazanavir is mixed with a water soluble polymer and/or a water insoluble polymer and extruded by hot melt granulation process, or melt granulation process.

16. The method according to claim 13, comprising preparing a substantially homogeneous melt of the ritonavir or atazanavir and optionally one or more excipients, extruding the melt, and cooling the melt until it solidifies.

17. The method according to claim 16, wherein the melt is formed at a temperature from substantially 50° C. to substantially 200° C.

18. The method according to claim 13, wherein the ritonavir, the polymer, and optionally one or more excipients are processed to form a powder blend which is transferred through the heated barrel of the extruder, whereby the powder blend melts and a molten solution product is formed, which is allowed to cool to form an extrudate.

19. The method according to claim 18, comprising processing the cooled extrudate into a desired pharmaceutical dosage form.

20. A pharmaceutical dosage form prepared by a method according to claim 19 in the form of a tablet or capsule.

21. The method according to claim 13, wherein the layer containing the atazanavir is prepared by direct compression or by wet granulation.

22. A composition prepared by a method according to claim 13 for use in the treatment of HIV or AIDS.
